(19) United States
(12) Patent Application Publication
(10) Pub. No.: US 2008/0260726 A1
(43) Pub. Date: Oct. 23, 2008

(54) Organic Compounds

(76) Inventors: Paul Andrew Whittaker, West Sussex (GB); Probir Chakravarty, West Sussex (GB); Alan Jackson, West Sussex (GB); Rosemary Sugar, West Sussex (GB)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/091,012

(22) PCT Filed: Oct. 19, 2006
(86) PCT No.: PCT/EP06/10096

§ 371 Date: Apr. 21, 2008

(30) Foreign Application Priority Data
Oct. 21, 2005 (GB) 0521512.4

(51) Int. Cl. A61K 39/395; C12Q 1/68; A61P 11/00

(52) U.S. Cl. 424/130.1; 435/6

(57) ABSTRACT

A method of identifying a substance suitable for use in inhibiting mucus hypersecretion that modulates the activity of a human cathepsin C gene or its gene product. The method involves combining a candidate substance with the human cathepsin C gene or its gene product and measuring the effect of the candidate substance on the activity of the gene or its gene product.

The present invention relates to the identification of substances or agents that modulate the activity of cathepsin C and the use of such substances in the treatment of diseases associated with abnormally high mucus secretion, particularly inflammatory or obstructive diseases of the respiratory system such as chronic obstructive pulmonary disease and asthma.

Mucus hypersecretion is a condition prevalent in patients suffering from chronic obstructive pulmonary disease (COPD) and asthma that is associated with excess mucus secretion in the airways. This can also involve the formation mucus plugs, increased susceptibility to infections, decline in lung function, morbidity and mortality. It is particularly problematic in COPD wherein mucus is fully released into airway lumen rather than “tethering” of mucus seen in asthma patients.

Currently there are no therapeutic approaches that demonstrably reduce mucus hypersecretion in COPD. There is therefore a need to develop drugs that inhibit mucus hypersecretion.

Mucus, which comprises heavily glycosylated apoproteins, is packaged in the secretory granules of goblet cells. Goblet cell mucins are thought to be important in mucus-related airways obstruction in COPD and asthma: Jackson A D (2001) Trends in Pharmacological Sciences 22:39-45; Rogers D F (2004) Current Opinion in Pharmacology 4:241-250; Kim S and Nadel J A (2004) Treatment in Respiratory Medicine 3:147-159].

It is proposed in accordance with the present invention that cathepsin C(CTSC) modulators inhibit secretion of mucus from goblet cells and as such are useful in the treatment of mucus hypersecretion. In fact CTSC inhibitors may have a dual effect as a mucus modulators and anti-inflammatories in respiratory disease as cathepsin C is involved in the activation of serine proteases in neutrophils and mast cells.

The present invention thus provides the CTSC gene and gene products as therapeutic targets for such diseases and an assay for identifying CTSC modulators i.e. candidate compounds or agents including peptides, peptidomimetics, small molecules or other drugs, which stimulate or inhibit the activity of CTSC or CTSC gene products, which may have therapeutic utility for inhibiting mucus hypersecretion associated with inflammatory and obstructive diseases of the respiratory system such as COPD and asthma.

In a first aspect the present invention relates to a method of identifying a substance suitable for use in inhibiting mucus hypersecretion that modulates the activity of a human cathepsin C gene or its gene product, wherein the method comprises combining a candidate substance with said gene or its gene product and measuring the effect of the candidate substance on the activity of said gene or its gene product.

Preferably the effect of the candidate substance on the activity of said gene or its gene product is measured with respect to the formation of goblet cells in human bronchial epithelial cells.

In a second aspect the present invention relates to a pharmaceutical composition comprising a compound that inhibits the human cathepsin C gene or its gene product, and a pharmaceutically acceptable carrier.

In a third aspect the present invention relates to the use of an antibody which is immunoreactive with a polypeptide encoded by a human cathepsin C gene, an antisense oligonucleotide (e.g. siRNA) comprising a nucleotide sequence complementary to a polynucleotide comprising a nucleotide sequence encoding that polypeptide, or a polynucleotide probe comprising at least 15 consecutive nucleotides of that polynucleotide, in the preparation of a pharmaceutical that inhibits mucus hypersecretion in human tissue.

In a fourth aspect the present invention relates to the use of an antibody which is immunoreactive with a polypeptide encoded by a human cathepsin C gene, an antisense oligonucleotide comprising a nucleotide sequence complementary to a polynucleotide comprising a nucleotide sequence encoding that polypeptide, or a polynucleotide probe comprising at least 15 consecutive nucleotides of that polynucleotide, in the preparation of a pharmaceutical for the treatment of an inflammatory or obstructive disease of the respiratory system. Preferably the inflammatory or obstructive disease is chronic obstructive pulmonary disease, asthma, cystic fibrosis or bronchiectasis.

In a fifth aspect the present invention relates to the use of a human cathepsin C inhibitor in the preparation of a pharmaceutical that inhibits mucus hypersecretion in human tissue, especially the lungs.

In a sixth aspect the present invention relates to the use of a human cathepsin C inhibitor in the preparation of a pharmaceutical for the treatment of an inflammatory or obstructive disease of the respiratory system.

Throughout this specification and in the claims that follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

As mentioned above, in a first aspect the present invention relates to a method of identifying a substance suitable for use in inhibiting mucus hypersecretion that modulates the activity of a human cathepsin C gene or its gene product, wherein the method comprises combining a candidate substance with said gene or its gene product and measuring the effect of the candidate substance on the activity of said gene or its gene product.

Cathepsins are a subclass of cysteine protease that belongs to the enzyme classification EC 3.4.22 (Barrett, A. J., N. D. Rawlings, et al., Handbook of Proteolytic Enzymes. London, Academic Press). They are known to play a major role in lysosomal, endosomal, and extracellular protein degradation and have thus been implicated in many disease processes.

Cathepsin C is a lysosomal cysteine protease from the papain superfamily that is known to function as a post-translational processing enzyme. It is also known as dipeptidylpeptidase I (DPP1). Its discovery was reported by Gutman and Fruton in J. Biol. Chem. (1948) 174, 851-858. It has been shown to be involved in the activation of immune and certain inflammatory cells in mice. Loss of function mutations in the CTSC gene have been shown to give rise to Papillon-Lefevre and Haim-Munk syndromes in humans, which are both autosomal recessive disorders characterised in patients by palmoplantar keratosis and severe early-onset periodontitis.

Substances that are suitable for use in the treatment of mucus hypersecretion will tend to be inhibitors of human CTSC genes or human CTSC gene products.

The activity of a CTSC gene product may be measured, for example, by a cell-based method or screening assay that identifies compounds which have an inhibitory effect on the activity of human CTSC gene or its gene product, or by an appropriate reporter gene assay.

The abovementioned screening method may be carried out, for example, by preparing cells which express a CTSC polypeptide, e.g. insect, mammal or yeast cells and then incubating the resulting cells with the candidate substance to determine the inhibition of a functional activity of a CTSC polypeptide.

Assay formats that are suitable for screening CTSC modulators are known. Such formats are suitable for high-throughput screening (HTS) for instance using fluorescence-based methods such as a plate-formatted fluorescence-based enzyme assay using recombinant cathepsin C and a fluorogenic peptide substrate.

The present invention also relates to a pharmaceutical composition comprising a compound that inhibits the human cathepsin C gene or its gene product, and a pharmaceutically acceptable carrier.

One can use an antibody which is immunoreactive with the polypeptide encoded by a CTSC gene (herein a “human CTSC antibody”) or an antisense oligonucleotide comprising a nucleotide sequence complementary to the polynucleotide comprising a nucleotide sequence encoding that polypeptide (herein a “human CTSC antisense oligonucleotide”), to prepare pharmaceuticals that inhibit mucus hypersecretion in human tissue.

The aforementioned human CTSC antibodies and antisense oligonucleotides may be used to treat inflammatory and obstructive diseases of the respiratory system that are caused or exacerbated by mucus hypersecretion.

Human CTSC inhibitors, human CTSC antibodies and human CTSC antisense oligonucleotides are hereinafter alternatively referred to collectively as “agents of the invention”.

Inflammatory or obstructive airways diseases and conditions to which the present invention is applicable include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of cystic fibrosis, bronchiectasis (persistent and progressive dilation of bronchi or bronchioles as a consequence of inflammatory disease, obstruction, or congenital abnormality) and bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Other inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

A human CTSC polypeptide can be isolated using any suitable conventional method.

A human CTSC polynucleotide may be cDNA, genomic DNA or RNA and may be obtained using any suitable conventional method. For example it may be prepared from the nucleotides which it comprises by chemical synthesis, e.g. automated solid phase synthesis using known procedures and apparatus.

A human CTSC antibody may be a polyclonal or monoclonal antibody. Such antibodies may be prepared using conventional procedures. Methods for the production of polyclonal antibodies against purified antigen are well established (cf. Cooper and Paterson in Current Protocols in Molecular Biology, Ausubel et al. Eds., John Wiley and Sons Inc., Chapter 11). Human CTSC antibodies may be used to detect, or determine the level of expression of, human CTSC polypeptides, or to inhibit ligand/anti-ligand binding activities of human CTSC polypeptides.

A human CTSC antisense oligonucleotide may be DNA, an analogue of DNA such as a phosphorothioate or methylphosphonate analogue of DNA, RNA, an analogue of RNA, or a peptide nucleic acid (PNA). The antisense oligonucleotide may be synthesised by conventional methods, for example using automated solid phase techniques. It may be used to inhibit the expression of a human CTSC gene, where this is desired. Alternatively, a short interfering RNA (siRNA) can be used as a specific tool for targeted gene knockdown. RNA interference inhibits gene expression and can therefore be used to explore gene function. This technique is described by S. M. Elbashir et al in Methods 26 (2002) 199-213.

A human CTSC polynucleotide probe comprises at least 15 contiguous nucleotides of the aforementioned polynucleotide or a complement thereof. The probe may be cDNA, genomic DNA or RNA and can be synthesised by conventional methods. Usually it is a synthetic oligonucleotide comprising 15 to 50 nucleotides, which can be labelled, e.g. with a fluorophore, to provide a detectable signal. A human CTSC polynucleotide probe can be used to detect the presence or absence of a human CTSC gene, i.e. to detect genetic abnormality.

The effectiveness of an agent of the invention in inhibiting mucus hypersecretory conditions, for example in inflammatory airways diseases, may be demonstrated in an animal model, e.g. a mouse or rat model, of goblet cell metaplasia or acute mucus secretion, for example as described by Stevenson et al, Am. J. Physiol. Lung Cell Mol. Physiol. (2005) 288: L514-L522; Singer et al, Nature Med. (2004) 10:193-196.

The effectiveness of an agent of the invention in inhibiting inflammatory conditions, for example in inflammatory airways diseases, may be demonstrated in an animal model, e.g. a mouse or rat model, of airways inflammation or other inflammatory conditions, for example as described by Szarka et al, J. Immunol. Methods (1997) 202:49-57; Renzi et al, Am. Rev. Respir. Dis. (1993) 148:932-939; Tsuyuki et al., J. Clin. Invest. (1995) 96:2924-2931; and Cernadas et al (1999) Am. J. Respir. Cell Mol. Biol. 20:1-8.

The effectiveness of an agent of the invention in inhibiting or reversing a leukocyte-associated inflammatory disease such as COPD may be demonstrated in a model of the disease, e.g. a lipopolysaccharide-induced lung inflammation model in rat or mouse or models described by Durie et al., Clin. Immunol. Immunopathol. (1994) 73: 11-18; and Williams et al, Proc. Natl. Acad. Sci. USA (1992) 89: 9784-9788.

The agents of the invention are also useful as co-therapeutic agents for use in combination with other drug substances such as anti-inflammatory, bronchodilatory, antihistamine, decongestant or anti-tussive drug substances, in the treatment of obstructive or inflammatory airways diseases such as those mentioned hereinbefore, for example as potentiators of therapeutic activity of such drugs or as a means of reducing required dosaging or potential side effects of such drugs. An agent of the invention may be mixed with the other drug substance in a fixed pharmaceutical composition or it may be administered separately, before, simultaneously with or after the other drug substance.

Accordingly the invention includes a combination of an agent of the invention as hereinbefore described with an anti-inflammatory, bronchodilatory, antihistamine, decongestant or anti-tussive drug substance, said agent of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248; LTB4 antagonists such as BIIL 284, CP-195543, DPC11870, LTB4 ethanolamide, LY 293111, LY 255283, CGS025019C, CP-195543, ONO-4057, SB 209247, SC-53228 and those described in U.S. Pat. No. 5,451,700; LTD4 antagonists such include montelukast, pranlukast, zafirlukast, accolate, SR2640, Wy-48,252, ICI 198615, MK-571, LY-171883, Ro 24-5913 and L-648051; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; A2A agonists such as those described in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618 and WO 04/046083; adenosine A2B receptor antagonists such as those described in WO 02/42298; and beta-2 adrenoceptor agonists such as albuterol (salbutamol), metaproterenol, terbutaline, salmeterol fenoterol, procaterol, and especially, formoterol, carmoterol and pharmaceutically acceptable salts thereof, and compounds (in free or salt or solvate form) of formula I of WO 0075114, which document is incorporated herein by reference, preferably compounds of the Examples thereof, especially a compound of formula

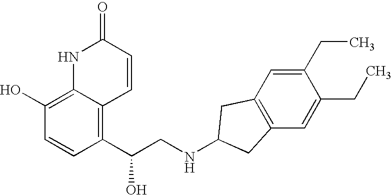

and pharmaceutically acceptable salts thereof, as well as compounds (in free or salt or solvate form) of formula I of WO 04/16601, and also compounds of EP 147719, EP 1440966, EP 1460064, EP 1477167, EP 1574501, JP 05025045, JP 2005187357, US 2002/0055651, US 2004/0242622, US 2004/0229904, US 2005/0133417, US 2005/5159448, US 2005/5159448, US 2005/171147, US 2005/182091, US 2005/182092, US 2005/209227, US 2005/256115, US 2005/277632, US 2005/272769, US 2005/239778, US 2005/215542, US 2005/215590, US 2006/19991, US 2006/58530, WO 93/18007, WO 99/64035, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618 WO 04/46083, WO 04/80964, WO 04/087142, WO 04/89892, WO 04/108675, WO 04/108676, WO 05/33121, WO 05/40103, WO 05/44787, WO 05/58867, WO 05/65650, WO 05/66140, WO 05/70908, WO 05/74924, WO 05/77361, WO 05/90288, WO 05/92860, WO 05/92887, WO 05/90287, WO 05/95328, WO 05/102350, WO 06/56471, WO 06/74897 or WO 06/8173.

Suitable bronchodilatory drugs include anticholinergic or antimuscarinic agents, in particular ipratropium bromide, oxitropium bromide, tiotropium salts and CHF 4226 (Chiesi), and glycopyrrolate, but also those described in EP 424021, U.S. Pat. No. 3,714,357, U.S. Pat. No. 5,171,744, WO 01/04118, WO 02/00652, WO 02/51841, WO 02/53564, WO 03/00840, WO 03/33495, WO 03/53966, WO 03/87094, WO 04/018422, WO 04/05285 and WO 05/077361.

Suitable dual anti-inflammatory and bronchodilatory drugs include dual beta-2 adrenoceptor agonist/muscarinic antagonists such as those disclosed in US 2004/0167167, US 2004/0242622, US 2005/182092, US 2005/256114, US 2006/35933, WO 04/74246, WO 04/74812, WO 04/89892 and WO 06/23475.

Suitable antihistamine drug substances include cetirizine hydrochloride, levocetirizine, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in JP 2004107299, WO 03/099807 and WO 04/026841.

Other useful combinations of agents of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770), and CCR-5 antagonists described in JP 2004359641, U.S. Pat. No. 6,166,037 (particularly claims 18 and 19), WO 00/66558 (particularly claim 8), WO 00/66559 (particularly claim 9), WO 04/018425 and WO 04/026873.

The agents of the invention may be administered by any appropriate route, e.g. orally, for example in the form of a tablet or capsule; parenterally, for example intravenously; topically, e.g. in an ointment or cream; transdermally, e.g. in a patch; by inhalation; or intranasally.

Pharmaceutical compositions containing agents of the invention may be prepared using conventional diluents or excipients and techniques known in the galenic art. Thus oral dosage forms may include tablets and capsules, and compositions for inhalation may comprise aerosol or other atomizable formulations or dry powder formulations.

When the composition comprises an aerosol formulation, it preferably contains, for example, a hydrofluoroalkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula I having a particle diameter up to 10 microns, optionally together with a diluent or carrier, such as lactose, of the desired particle size distribution and a compound that helps to protect against product performance deterioration due to moisture, such as magnesium stearate, e.g. 0.01 to 1.5%. When the composition comprises a nebulised formulation, it preferably contains, for example, the compound of formula I either dissolved, or suspended, in a vehicle containing water, a co-solvent such as ethanol or propylene glycol and a stabiliser, which may be a surfactant.

The invention includes (i) an agent of the invention in inhalable form, e.g. in an aerosol or other atomizable composition or in inhalable particulate, e.g. micronised form, (ii) an inhalable medicament comprising an agent of the invention in inhalable form; (iii) a pharmaceutical product comprising such an agent of the invention in inhalable form in association with an inhalation device; and (iv) an inhalation device containing an agent of the invention in inhalable form.

Dosages of agents of the invention employed in practising the present invention may of course vary depending, for example, on the particular condition to be treated, the effect desired and the mode of administration. In general, suitable daily dosages for administration by inhalation are of the order of 1 μg to 10 mg/kg while for oral administration suitable daily doses are of the order of 0.1 mg to 1000 mg/kg.

The invention is illustrated by the following Examples. Abbreviations used in the Examples have the following meanings:

A adenosine
ALI air-liquid interface
ATP adenosine triphosphate
bp base pair
CO₂ carbon dioxide
cRNA complementary ribonucleic acid
CTSC cathepsin C
C cytosine
DMSO dimethyl sulphoxide
ds-cDNA double-stranded cDNA
ELLA enzyme-linked lectin assay
G guanosine
HBEC human bronchial epithelial cell
IL13 interleukin 13
IMS industrial methylated spirit
LF lipofectamine 2000
mRNA messenger ribonucleic acid
NBF neutral buffered formalin
P phosphate
pbs phosphate buffered saline
P2 passage 2
RNA ribonucleic acid
RT-PCR reverse transcription polymerase chain reaction
SDS sodium dodecyl sulphate
siRNA small interfering ribonucleic acid
U uracil

### EXAMPLE 1

### Identification of CTSC by “Genechip” Profiling

It is known that cultured primary human bronchial epithelial cells (HBECs) can be stimulated to differentiate into a multicellular layer containing mucus-producing goblet cells over a 14 day period when cultured at air-liquid interface: Atherton et al in Am. J. Physiol. Lung Cell Mol. Physiol. (2003) 285: L730-L739. The numbers of goblet cells can be manipulated using physiological and pharmacological approaches. This finding is used as the basis for an experimental strategy to identify genes whose expression correlates with goblet cell formation in the HBEC model by Affymetrix chip profiling.

HBECs (donors 1F1811 and 2F1578; obtained from Cambrex Bio Science) at passage 2 (P2) are seeded into 12 well Transwell Clear inserts (Corning) at 8.25×10⁴ cells per insert, maintained as submerged cultures for 7 days, then exposed to an air-liquid interface (ALI) for 14 days as described in Atherton et al. Am. J. Physiol. Lung Cell Mol. Physiol. (2003) 285: L730-L739. Goblet cell density in the resulting multilayered bronchial epithelial tissue is modulated by either varying the concentration of Interleukin 13 (IL13; 1 ng/ml and 10 ng/ml), removing epidermal growth factor from the differentiation medium, or addition of the p38 MAP kinase inhibitors SB-202190 (Tocris) and AAZ102 to 1 μM and 10 μM to the differentiation medium in the presence of 1 ng/ml IL13. Changes in goblet cell density in each of these conditions is compared against the basal level of differentiation in the absence of IL13. Samples of total RNA from 6 inserts of cells at days 0, 7 and 14 ALI are prepared by lysis of cells on inserts using TRIZOL® reagent (Invitrogen) extraction, DNase I treatment and purification using RNeasy® mini spin columns (Qiagen).

Genome wide RNA transcript profiling of the isolated RNA samples is performed using Affymetrix U133A GeneChip expression probe arrays. RNA samples are used as a template for synthesis of double-stranded cDNA (ds-cDNA) in the presence of a T(24)T7 primer. The tailed ds-cDNA is purified using an affinity resin column and second-strand synthesis is performed by in vitro transcription using T7 RNA polymerase in the presence of biotinylated ribonucleotides. After RNeasy purification, the labelled cRNA is hybridized against Affymetrix U133A GeneChips overnight, followed by washing and staining with streptavidin-coupled phycoerythrin. Each GeneChip is scanned, the images are processed using the Microarray Analysis Suite (Affymetrix) version 5 (MASS), and the resulting files (.CEL files) are up-loaded to a database. All expression experiments are scaled to the same target intensity (150).

The expression data is downloaded into GeneSpring 6.2 (Silicon genetics, Inc.) for data visualisation and the generation of lists of probesets from comparisons of gene expression profiles of HBECs at different timepoints (e.g. day 7) and under different experimental conditions (e.g. 1 ng IL-13). In order to generate lists of probesets enriched for genes involved in goblet cell formation and mucus secretion two approaches are taken.

In the first approach, a non-redundant list of 1387 probesets is generated by comparison of the lists of probesets between sequential timepoints (i.e. day 0 and day 7, day 7 and day 14) and time-matched timepoints (e.g. day 7 1 ng IL13 per ml versus day 7 vehicle) for each of the experimental conditions. This non-redundant list is further reduced to a list of 185 probesets using a scoring system in which individual genes on the list are scored according to 12 criteria (e.g. up regulation in either 1 ng or 10 ng IL13 at day 7 compared to vehicle 0). The basis of the scoring system is to reward probesets with a desirable profile (e.g. up regulation with IL13) and penalise probesets with undesirable profiles (e.g. not up regulated with IL13). The twelve scoring criteria applied are:

A. Up regulation in either 1 ng or 10 ng IL13 at day 7 compared to vehicle 0
A fold change of >2 fold compared to vehicle was designated as scoring 2
A fold change of <2 fold compared to vehilce was designated as scoring 0
A fold change of <1 fold compared to vehicle was designated as scoring −1
B. Up regulation in 1 ng or 10 ng at day 7 compared to vehicle 7 (time matched control)
A fold change of >2 fold compared to vehicle was designated as scoring 2
A fold change of <2 fold compared to vehilce was designated as scoring 0
A fold change of <1 fold compared to vehicle was designated as scoring −1
C. Up regulation in 1 ng IL13 at day 14 compared to vehicle 14 (time matched control)
A fold change of >2 fold compared to vehicle was designated as scoring 2
A fold change of <2 fold compared to vehilce was designated as scoring 0
A fold change of <1 fold compared to vehicle was designated as scoring −1
D. Down regulation in 10 ng IL13 at day 14 compared to vehicle 14 (time matched control)
A fold change of <2 fold compared to vehicle was designated as scoring 2
A fold change of <−1 fold compared to vehicle was designated as scoring −1
A fold change of >2 fold compared to vehicle was designated as scoring −1
E. Up regulation in 1 ng IL13 at day 14 compared to 1 ng IL13 at day 7
A fold change of >2 fold compared to vehicle was designated as scoring 2
A fold change of <2 fold compared to vehilce was designated as scoring 1
A fold change of <−2 fold compared to vehicle was designated as scoring −1
F. Down regulation in 10 ng IL13 at day 14 compared to 10 ng IL13
A fold change of <2 fold compared to vehicle was designated as scoring 2
A fold change of <−1 fold compared to vehicle was designated as scoring −1
A fold change of >2 fold compared to vehicle was designated as scoring −1
G. Exaggerated in 2F1578 donor compared to 1F1887 donor (1 ng IL13 at day 7)
A fold change of >2 fold compared to vehicle was designated as scoring 2
A fold change of <2 fold compared to vehilce was designated as scoring 0
A fold change of <1 fold compared to vehicle was designated as scoring −1
H. Exaggerated in 2F1578 donor compared to 1F1887 donor (1 ng IL13 at day 14)
A fold change of >2 fold compared to vehicle was designated as scoring 2
A fold change of <2 fold compared to vehilce was designated as scoring 0
A fold change of <1 fold compared to vehicle was designated as scoring −1
I. Down regulation in 1 ng IL13 +10 uM SB at day 7 compared with 1 ng IL13 day 7
A fold change of <2 fold compared to vehicle was designated as scoring 2
A fold change of <−1 fold compared to vehicle was designated as scoring −1
A fold change of >2 fold compared to vehicle was designated as scoring −1
J. Down regulation in 1 ng IL13 +10 uM SB at day 14 compared with 1 ng IL13 day 14
A fold change of <2 fold compared to vehicle was designated as scoring 2
A fold change of <−1 fold compared to vehicle was designated as scoring −1
A fold change of >2 fold compared to vehicle was designated as scoring −1
K. Down regulation in 1 ng IL13 +1 uM AAZ at day 7 compared with 1 ng IL13 day 7
A fold change of <2 fold compared to vehicle was designated as scoring 2
A fold change of <−1 fold compared to vehicle was designated as scoring −1
A fold change of >2 fold compared to vehicle was designated as scoring −1
L. Down regulation in 1 ng IL13 +1 uM AAZ at day 14 compared with 1 ng IL13 day 14
A fold change of <2 fold compared to vehicle was designated as scoring 2
A fold change of <−1 fold compared to vehicle was designated as scoring −1
A fold change of >2 fold compared to vehicle was designated as scoring −1

Scores are summed across all tests and the genes are ranked based on the final score. Scores ranging from −15 to +37 are obtained and the threshold for cut off is determined to be +20, giving a list of 185 probesets. The list is further reduced to 110 probesets by removing all genes which show less than a 2-fold up regulation.

In the second approach, the list of probesets is generated using only day 7 time-matched data from donor 2F1578 and identifying probesets for genes which are greater than 2-fold up regulated with 1 ng per ml IL13, but down regulated with 10 μM SB20219 in the presence of IL13. Fifty probesets fulfilling this criteria are identified.

Comparison of the lists of probesets generated by the 2 approaches shows that 44 of the 50 probesets generated by Approach 2 are contained within the list of probesets identified by Approach 1. Thus a list of 116 probesets corresponding to 93 genes potentially involved in goblet cell formation/mucus secretion is identified.

The list of 93 genes is further refined using a combination of bioinformatics annotation (gene ontology classification, pathway assignment), literature annotation (biological function) and subjective assessment (drugability, assayability, biological role) to produce a list of 10 genes for experimental validation of a role in goblet cell formation and/or mucus secretion using gene knockdown and small molecule inhibitor experiments in vitro.

### EXAMPLE 2

### Identification that Inhibition of CTSC Gene Expression or Protein Activity Inhibits Goblet Cell Formation

The effect of knocking down cathepsin C gene expression on goblet cell formation in IL13-differentiated HBECs is examined using double-stranded small interfering RNAs (siRNAs). Using Refseq sequence NM_{—}001814, 4 siRNA duplexes targeting the cathepsin C gene and consisting of a 19 bp RNA duplex with 2 nucleotide overhang at the 3′-end are designed and synthesised by Dharmacon Inc:

Duplex 1:

(SEQ ID.01)

GGAGAAAUGUUCAUGGUAUUU (sense strand)

(SEQ ID.02)

5′-P-AUACCAUGAACAUUUCUCCUU (antisense strand)
Duplex 2:

(SEQ ID.03)

GCAAUGAAGCCCUGAUGAAUU (sense strand)

(SEQ ID.04)

5′-P-UUCAUCAGGGCUUCAUUGCUU (antisense strand)
Duplex 3:

(SEQ ID.05)

CUAAUAGGCUCUACAAGUAUU (sense strand)

(SEQ ID.06)

5′-P-UACUUGUAGAGCCUAUUAGUU (antisense strand)
Duplex 4:

(SEQ ID.07)

CCUUAAGAAUUCUCAGGAAUU (sense strand)

(SEQ ID.08)

5′-P-UUCCUGAGAAUUCUUAAGGUU (antisense strand)

The siRNA duplexes are transfected into HBECs using Lipofectamine 200™. Transfection medium is prepared by mixing OPTI-MEM® I reduced serum medium (Invitrogen Corp.), siRNA duplex (5× final concentration) and Lipofectamine 2000™ (5× final concentration), incubating at room temperature for 30 minutes, then diluting 1:5 in antibiotic free differentiation medium (DIFF-AB medium; bronchial epithelial basal medium and Dulbeccos modified eagles medium [50:50] plus bronchial epithelial growth medium SingleQuots [bovine pituitary extract, insulin, hydrocortisone, all-trans retinoic acid, transferrin, epinephrine, tri-iodothyronine and human epidermal growth factor at the recommended concentrations, but no tri-iodothyronine and gentamycin/amphotericin B], all obtained from BioWhittaker) prior to addition to HBECs. For these experiments an abbreviated version of the HBEC model is used in which HBECs are maintained as submerged cultures for 3 days and then transferred to ALI for 6 days. Cells from a passage 2 (P2) stock of HBEC donor 2F1578 are cultured as described in Atherton et al. Am. J. Physiol. Lung Cell Mol. Physiol. (2003) 285: L730-L739 to approximately 85% confluence before harvesting for transfection. After trypsinisation and recovery by centrifugation, cells are resuspended at a density of 16.5×10⁴ cells per ml in DIFF-AB medium containing siRNA duplex (10 nM) and Lipofectamine 2000™ (1 μl per ml) and seeded onto 12 well Transwell Clear inserts. One ml of HBEC/DIFF-AB medium/siRNA/lipofectamine 2000™ mix is deposited onto each insert and 1 ml of DIFF-AB medium is added to the basolateral chamber of each insert. After 24 hours (day 1 of submerged culture), all media is aspirated (apically and basolaterally) and replaced with 0.5 ml apically and 1 ml basolaterally of DIFF-AB medium containing 50 μg per ml gentamycin but no amphotericin B (DIFF-AB+G). On day 3, DIFF-AB+G medium is aspirated from the apical and basolateral surfaces and HBECs are cultured at ALI with DIFF-AB+G medium containing 1 ng per ml IL13. All cells are incubated at 37° C., 5% CO₂ and 95% relative humidity. The basolateral medium is aspirated at days 5 and 7 and replaced with 1 ml DIFF-AB+G medium containing IL13. In addition the apical surface is rinsed with 0.5 ml phosphate buffered saline (PBS) to remove accumulated cell debris/mucus. Cells are harvested for total RNA extraction at days 3, 5, 7 and 9 as described above, and for goblet cell analysis by histology at day 9. For histology, inserts are fixed by the addition of 0.5 ml 10% neutral buffered formalin (NBF) to the apical surface followed by 1 ml NBF to the basolateral surface. After 24 hours of fixation the inserts are bisected and processed to RALwax through graded industrial methylated spirit (IMS) and chloroform. Both halves of the insert are embedded on edge using fresh RALwax. The paraffin blocks are sectioned at 45 μm and stained with 45M1 antibody (an anti-MUCSAC antibody obtained from Lab-Vision Neomarkers) and alcian blue/haematoxylin and eosin (AB/H&E) according to standard histological protocols to enable detection of mucin and discrimination of individual cells by nuclear staining. The stained slides are examined microscopically.

CTSC siRNA duplexes 1-4 are found to down regulate CTSC gene expression in transfected cells either individually or in combination (SMARTPOOL containing 2.5 nM of each duplex giving 10 nM total) relative to cells treated with IL13, but not exposed to transfection reagents. CTSC levels are measured by RT-PCR of total RNA extracted from HBECs by TaqMan® (Applied BioSystems) using the following primer pair and probe set:

| | |
|---|---|
| FORWARD PRIMER: | |
| 5′-AGATATGATTAGGAGAAGTG-3′ | (SEQ ID.09) |
| REVERSE PRIMER: | |
| 5′-TCTTTTGCTGTATTTCAGCAGTCAGT-3′ | (SEQ ID.10) |
| Probe: | |
| 5′-FAM-ATCCCAAGGCCCAAAC-3′ | (SEQ ID.11) |

The Table 1 below shows the results of one experiment where the mRNA levels as measured by TaqMan® (Applied Biosystems) analysis are expressed as a percentage of the levels in cells treated with IL13 alone and not transfected:

TABLE 1

| Sample | Day 3* | Day 5* | Day 7* | Day 9* |
|---|---|---|---|---|
| HBECs + IL13 alone | 100.00 (5.10) | 100.00 (3.53) | 100.00 (9.61) | 100.00 (3.94) |
| HBECs + 1 ul LF | 97.83 (5.87) | 91.30 (18.29) | 89.80 (5.01) | 96.36 (19.06) |
| 2000 ™ | | | | |
| CSTC duplex 1 | 18.05 (1.23) | 21.55 (2.11) | 26.90 (0.83) | 23.46 (2.29) |
| CSTC duplex 2 | 20.54 (1.19) | 24.63 (4.38) | 38.70 (1.75) | 47.66 (3.32) |
| CSTC duplex 3 | 22.69 (0.00) | 33.13 (9.02) | 23.79 (0.57) | 27.47 (2.47) |
| CSTC duplex 4 | 41.57 (2.60) | 32.37 (4.04) | 35.82 (2.76) | 51.81 (4.87) |
| CSTC SmartPool | 28.26 (1.52) | 25.46 (0.55) | 30.92 (0.97) | 44.88 (3.88) |
| Cyclophilin siCONTROL | 104.88 (6.92) | 96.69 (6.24) | 91.42 (5.39) | 126.20 (10.08) |

*standard error of the mean shown in brackets

Knockdown of CTSC mRNA levels is seen with all 4 siRNA duplexes over the 9 day culture period compared with untransfected cells. Duplexes 1 and 3 most efficiently knockdown CTSC mRNA levels. CTSC mRNA levels in cells treated with lipofectamine 2000™ alone, or with cyclophilin B siRNA (Dharmacon Inc.) are not significantly altered.

The effects of CTSC knockdown on goblet cell formation are investigated by measuring 45M1-stained area (μm²/μm of epithelium) and expressed as a percentage of IL13 treated cells. The results are shown in Table 2 below:

TABLE 2

| Sample | 45M1 Stained Area Day 9* |
|---|---|
| HBECs + IL13 alone | 100 (31.485) |
| HBECs + 1 ul LF 2000 ™ | 82.770 (15.102) |
| CTSC duplex 1 | 4.014 (2.516) |
| CTSC duplex 2 | 49.870 (13.784) |
| CTSC duplex 3 | 19.692 (4.084) |
| CTSC duplex 4 | 24.274 (11.158) |
| CTSC SmartPool | 27.702 (4.664) |
| Cyclophilin siCONTROL | 100.908 (12.404) |

*standard error of the mean shown in brackets

Each of the 4 siRNA duplexes (plus the SmartPool) significantly reduces the area of MUCSAC staining in the epithelia indicating that reduction of CTSC gene expression over the 9 day timecourse inhibits goblet cell formation as measured by 45M1 and indicating a functional role for CTSC in goblet cell formation.

Duplex 1 and 3 (identified as the most effective siRNA duplexes) concentration-dependently knock down CTSC mRNA over a 9 day period to levels below those observed in untransfected cells. Results obtained for duplex 1 are shown in Table 3 below:

TABLE 3

| Sample | Day 5* | Day 7* | Day 9* |
|---|---|---|---|
| Untreated − IL13 | 77.71 (16.5) | 40.17 (1.4) | 43.22 (2.99) |
| Untreated + IL13 | 100.00 (1.23) | 100.00 (6.55) | 100.00 (5.74) |
| LF + IL-13 | 91.00 (3.2) | 97.29 (3.14) | 100.23 (1.85) |
| CTSC duplex 3 | 14.04 (0.93) | 18.72 (0.35) | 30.32 (2.18) |
| (10 nM) | | | |
| CTSC duplex 3 (3 nM) | 22.32 (0.97) | 35.64 (6.91) | 46.54 (1.52) |
| CTSC duplex 3 (1 nM) | 34.52 (3.09) | 51.71 (4.14) | 60.27 (2.28) |
| CTSC duplex 3 | 52.21 (4.32) | 68.17 (5.42) | 92.62 (1.32) |
| (0.3 nM) | | | |
| Cyclophilin | 113.38 (11.3) | 97.89 (9.5) | 105.64 (8.14) |
| siCONTROL (10 nM) | | | |

*standard error of the mean shown in brackets

As seen in previous experiments, the levels of CTSC mRNA in cells treated with lipofectamine 2000™ alone, or with cyclophilin B siRNA (Dharmacon Inc.) are not significantly altered. In addition, the siRNA duplexes dose-dependently reduce 45M1 staining area by immunohistochemistry in epithelial sections and MUCSAC levels as measured by TaqMan relative to untransfected cells. The results are shown in Table 4 below:

TABLE 4

| Sample | Histology Day 9* | MUC5AC D9* |
|---|---|---|
| Untreated − IL13 | 12.094 (4.205) | 29.39 (7.26) |
| Untreated + IL13 | 100.000 (7.897) | 100.00 (1.72) |
| LF + IL-13 | 132.413 (37.797) | 71.46 (4.37) |
| CTSC duplex 3 10 nM | 39.714 (2.857) | 23.17 (2.55) |
| CTSC duplex 3 3 nM | 45.691 (14.656) | 30.98 (1.84) |
| CTSC duplex 3 1 nM | 100.420 (16.096) | 71.17 (1.87) |
| CTSC duplex 3 0.3 nM | 78.919 (21.534) | 122.77 (12.34) |
| Cyclophilin siCONTROL 10 nM | 151.067 (7.765) | 112.60 (12.09) |

*standard error of the mean shown in brackets

The effect of siRNA knockdown of gene expression on CTSC protein levels is measured by western blotting of lysates from HBECs treated with siRNA duplexes 1 and 3 at days 5, 7 and 9 using an affinity purified goat polyclonal antibody raised against a peptide mapping near the carboxy terminus of cathepsin C of human origin (antibody T17 [sc-S647] from Santa Cruz Biotechnology Inc.). For protein preparation, the inserts are washed basolaterally (x1) and apically (x2) with 1 ml cold PBS and 2 inserts of HBECs from each timepoint/siRNA concentration/control are lysed for 30 min at 4° C. in 75 μl of 50 mM Tris/150 mM NaCl/20 mM EDTA pH7.6/1% Triton containing phosphatase inhibitor cocktail II (Calibiochem) and protease inhibitor cocktail III (Calibiochem) diluted to 1:100. The lysates from both inserts are recovered by scraping into a microcentrifuge tube and the lysate is centrifuged at 16,000 g for 5 min. The supernatant is recovered and 17 μl aliquots are analysed on a 4-12% Tris-Bis NuPAGE™ gel (Invitrogen) using NuPAGE MES SDS running buffer. After electrophoresis, the gel is western blotted onto HyBond ECL nitrocellulose (Amersham) using standard protocols for the NuPAGE system (Invitrogen). The blot is blocked with Odyssey blocking buffer (LI-COR Biosciences Ltd.) for 60 min at room temperature and incubated with a 1:100 dilution of T17 antibody for 60 min at room temperature. After washing 4 times for 10 min in PBS containing 0.05% Tween-20 at room temperature, the blot is incubated with a 1:2500 dilution of anti-goat Alexa Fluor 680 secondary antibody (Invitrogen), Protein bands hybridising to the T17 antibody are visualised and quantified at 700 nm at a resolution of 84 μm using the LI-COR Odyssey Infrared Imager (LI-COR Biosciences Ltd.). The antibody detects a protein band of around 7 kDa (corresponding to the light chain of CTSC) on the western blots in IL13-treated HBECs which increases in intensity from day 5 to day 9. In addition to the 7 kDa band the T17 antibody detects several other higher molecular weight bands which do not change in intensity with IL13 treatment, One of these invariant bands is used as an internal control so that the intensity of the 7 kDa band is expressed as a ratio compared with this. The band is reduced in intensity in lysates from siRNA-treated HBECs at siRNA concentrations of 10 nM, 3 nM and 1 nM at days 5, 7 and 9. At 0.3 nM siRNA, knockdown is seen at day 5, but progressively increases in amount on days 7 and 9. The results are shown in Table 5 below:

TABLE 5

| | | | | | 3 nM | 1 nM | 0.3 nM | |
|---|---|---|---|---|---|---|---|---|
| | | | Plus | 10 nM | CTSC | CTSC | CTSC | 10 nM |
| | Minus | Plus | Il-13 + 1 ul | CTSC | duplex | duplex | duplex | Cyclophilin B |
| Day | IL-13 | Il-13 | Lf | duplex #3 | #3 | #3 | #3 | siControl |
| 5 | 0.00513 | 0.33841 | 0.11091 | 0.03110 | 0.06231 | 0.04574 | 0.08696 | 0.14954 |
| 7 | 0.06948 | 0.54964 | 0.42848 | 0.03886 | 0.11707 | 0.05304 | 0.11214 | 0.27292 |
| 9 | 0.09173 | 1.01773 | 0.75133 | 0.05773 | 0.09063 | 0.10916 | 0.23379 | 0.25677 |

The broad spectrum cathepsin inhibitor propane-1-sulfonic acid {4-[2-cyano-7-(2,2-dimethyl-propyl)-7H-pyrrolo[2,3-d]pyrimidin-6-ylmethyl]-phenyl}-amide (herein “Compound A”) is tested for its effect on goblet cell formation in the 9 day model by adding the compound to the basolateral medium at 0.1, 1 and 10 μM on days 3, 5 and 7. The effect on goblet cell formation is examined histologically by 45M1 and AB/PAS staining per unit length of epithelium. The results are given in Table 6 below:

TABLE 6

| | | | Compd A | Compd A | Compd A |
|---|---|---|---|---|---|
| | | Compd A | 10 uM + | 1 uM + | 0.1 uM + |
| Vehicle* | IL13* | 10 uM* | IL13* | IL13* | IL13* |
| 0.545 | 2.429 | 0.300 | 1.424 | 2.621 | 2.533 |
| (0.196) | (0.075) | (0.055) | (0.166) | (0.257) | (0.218) |

*standard error of the mean shown in brackets

A modest effect at 10 μM is seen, with no effect at 0.1 and 1 μM. Treatment of cells for longer (11 days rather than 6 days) at ALI shows a greater inhibition at 10 μM. The results are shown in FIG. 7 below:

TABLE 7

| | | | Compd A | | Compd A |
|---|---|---|---|---|---|
| | IL-13 | Compd A | 10 uM + | Compd A | 0.1 uM + |
| Vehicle | 1 ng/ml | 10 uM | IL-13 | 1 uM + IL-13 | IL-13 |
| 0.154 | 4.479 | 0.153 | 0.886 | 3.700 | 3.609 |
| (0.055) | (0.209) | (0.040) | (0.150) | (0.518 | (0.361) |

*standard error of the mean shown in brackets

### EXAMPLE 3

### Identification that Inhibition of CTSC Gene Expression or Protein Activity Inhibits Mucus Secretion

The effect of siRNA knockdown of CTSC expression on mucin secretion in differentiated HBECs is examined using the CTSC SMARTPOOL at different concentrations (0.3-10 nM). Adenosine triphosphate is used as a mucin secretagogue and secreted mucin is measured using an enzyme-linked lectin assay (ELLA; Kemp, Sugar and Jackson Am. J. Respir. Cell Mol. Biol. (2004) 31: 446-455). HBECs (16.5×10⁴ cells per insert from donor 2F1S78) are transfected and cultured using the 9 day differentiation model as described in Example 2. At day 9 (6 days ALI) HBEC inserts are exposed to 100 μM ATP-γS for 10 minutes and supernatants are removed for analysis by ELLA. ATP-γS-induced mucus secretion is inhibited at all CTSC siRNA SMARTPOOL concentrations, but is unaffected by either cyclophilin B siRNA or Lipofectamine alone. The results are shown in Table 8 below:

TABLE 8

| | | | | CTSC | CTSC | CTSC | CTSC |
|---|---|---|---|---|---|---|---|
| | | LF 2000 | Cyclophilin | siRNA | siRNA | siRNA | siRNA |
| Vehicle* | IL-13 alone* | (1 μl)* | B siRNA* | 10 nM* | 3 nM* | 1 nM* | 0.3 nM* |
| 419.74 | 634.55 | 779.19 | 486.17 | 109.28 | 165.34 | 136.86 | 296.67 |
| (102.46) | (95.65) | (92.63) | (58.74) | (26.63) | (39.58) | (15.29) | (28.95) |

*standard error of the mean shown in brackets

The effect of Compound A on mucus secretion in the 9 day model is examined by adding the compound at various concentrations (0.1-10 μM) to the apical surface in minimal medium containing 0.1% dimethyl sulphoxide (DMSO) and incubating for 30 minutes at 37° C., followed by 10 minutes exposure to 100 μM ATP-γS. Compound A dose-dependently inhibits mucus secretion. The results are shown in Table 9 below:

TABLE 9

| | ATP | Compd A | Compd A | Compd A | Compd A |
|---|---|---|---|---|---|
| Vehicle | (100 μM) | (10 μM) only | (10 μM) | (10 μM) | (0.1 μM) |
| 100.00 | 214.15 | 88.99 | 120.18 | 177.02 | 203.51 |
| (9.84) | (17.79) | (11.93) | (7.35) | (20.93) | (13.86) |

*standard error of the mean shown in brackets

1. A method of identifying a substance suitable for use in inhibiting mucus hypersecretion that modulates the activity of a human cathepsin C gene or its gene product, wherein the method comprises combining a candidate substance with said gene or its gene product and measuring the effect of the candidate substance on the activity of said gene or its gene product.

2. A method according to claim 1 wherein the human cathepsin C gene product is a cathepsin C polypeptide.

3. A method according to claim 1 wherein the effect of the candidate substance on the activity of said gene or its gene product is measured with respect to the formation of goblet cells in human bronchial epithelial cells.

4. A pharmaceutical composition comprising a compound that inhibits the human cathepsin C gene or its gene product, and a pharmaceutically acceptable carrier.

5. A pharmaceutical composition according to claim 4 that contains another drug substance which is an anti-inflammatory, a bronchodilator, an antihistamine, a decongestant or an anti-tussive drug substance, optionally together with a pharmaceutically acceptable diluent or carrier.

6. The use of an antibody which is immunoreactive with a polypeptide encoded by a human cathepsin C gene, an antisense oligonucleotide comprising a nucleotide sequence complementary to a polynucleotide comprising a nucleotide sequence encoding that polypeptide, or a polynucleotide probe comprising at least 15 consecutive nucleotides of that polynucleotide, in the preparation of a pharmaceutical that inhibits mucus hypersecretion in human tissue.

7. The use of an antibody which is immunoreactive with a polypeptide encoded by a human cathepsin C gene, an antisense oligonucleotide comprising a nucleotide sequence complementary to a polynucleotide comprising a nucleotide sequence encoding that polypeptide, or a polynucleotide probe comprising at least 15 consecutive nucleotides of that polynucleotide, in the preparation of a pharmaceutical for the treatment of an inflammatory or obstructive disease of the respiratory system.

8. The use of a human cathepsin C inhibitor in the preparation of a pharmaceutical that inhibits mucus hypersecretion in human tissue.

9. The use of a human cathepsin C inhibitor in the preparation of a pharmaceutical for the treatment of an inflammatory or obstructive disease of the respiratory system.

10. The use according to claim 9 wherein the inflammatory or obstructive disease is chronic obstructive pulmonary disease, asthma, cystic fibrosis or bronchiectasis.
